(19) United States
(12) Patent Application Publication
(10) Pub. No.: US 2023/0226027 A1
(43) Pub. Date: Jul. 20, 2023

(54) THIENOPYRIDINE DERIVATIVES FOR USE IN THE TREATMENT OF CORONAVIRUS INFECTION

(71) Applicants: INSERM (INSTITUT NATIONAL DE LA SANTÉ ET DE LA RECHERCHE MÉDICALE), Paris (FR); ECOLE NATIONALE VÉTÉRINAIRE DE TOULOUSE, Toulouse (FR); INSTITUT NATIONAL DE RECHERCHE POUR L'AGRICULTURE, L'ALIMENTATION ET L'ENVIRONNEMENT, Paris (FR); UNIVERSITÉ TOULOUSE III - PAUL SABATIER, Toulouse (FR)

(72) Inventors: Frank LEZOUALC'H, Toulouse (FR); Romain VOLMER, Toulouse (FR); Charlotte FORET-LUCAS, Toulouse (FR); Thomas FIGUEROA, Toulouse (FR)

(21) Appl. No.: 18/001,147

(22) PCT Filed: Jun. 11, 2021
(86) PCT No.: PCT/EP2021/065756

§ 371 Date: Dec. 8, 2022

(30) Foreign Application Priority Data
Jun. 12, 2020 (EP) 20305645.2

(51) Int. Cl. A61K 31/4365; A61P 31/14

(52) U.S. Cl. CPC A61K 31/4365; A61P 31/14

(57) ABSTRACT

Coronaviridae is a family of enveloped, positive-sense, single-stranded RNA viruses. The emergence of a new beta-coronavirus SARS-CoV-2 has led to a major health-related crisis associated with a significant mortality in intensive care units, due to the pulmonary complications of COVID-19. The inventors showed that an inhibitor of EPAC1 (i.e. AM-001) is suitable for inhibiting replication of coronavirus and thus would be suitable for the treatment of infections mediated by said type of virus.

### FIELD OF THE INVENTION

The present invention is in the field of medicine, and in particular virology.

### BACKGROUND OF THE INVENTION

The Severe Acute Respiratory Syndrome coronavirus 2 (SARS-CoV-2) which emerged in Wuhan, China, in December 2019 induced a threat to global health. In Mar. 11, 2020, the WHO declared COVID-19 as a pandemic. The rapidity, rate of global spread and observed enhanced mortality raises public health, socio-economic and scientific challenges. As of yet, as it seems to spread very actively, it has infected more than 185 countries with more than 4,100, 000 confirmed cases, and more than 280,000 deaths as of May 10, 2020. SARS-CoV-2 can cause a respiratory syndrome that manifests a clinical pathology resembling mild upper respiratory tract disease (common cold-like symptoms) and occasionally severe lower respiratory tract illness and extra-pulmonary manifestations leading to multi-organ failure and death. This pandemic follows several highly pathogenic human coronaviruses infections including SARS-CoV in 2002 with a death rate of 10% and MERS-CoV in 2012 with a death rate of 36%. No treatment is available. To fight against the COVID-19 pandemic in a long term, in addition to the containment measures implemented in many countries, several projects have been launched around the world to understand the viral evolution and the pathophysiological consequences of the infection in order to identify therapeutic targets and to implement innovative therapies.

### SUMMARY OF THE INVENTION

As defined by the claims, the present invention relates to methods for the treatment of coronavirus infections.

### DETAILED DESCRIPTION OF THE INVENTION

The present invention is based in the fact that the pharmacological inhibition of Epac1 by AM001 significantly decreases the replication of the SARS-Cov-2.

Accordingly, the first object of the present invention relates to a method of treating a coronavirus infection in a subject in need thereof comprising administrating to the subject a therapeutically effective amount of a compound having the following formula (I):

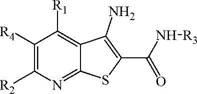

wherein:
R₁ is selected from the group consisting of:
   H;
   (C₂-C₂₀)alkyl;
   (C₃-C₁₀)cycloalkyl;
   3-10 membered heterocycloalkyl;
   (C₆-C₁₀)aryl; and
   5-10 membered heteroaryl;
wherein said alkyl, cycloalkyl, heterocycloalkyl, aryl and heteroaryl groups are optionally substituted;
R₂ is selected from the group consisting of:
   H;
   (C₁-C₂₀)alkyl;
   (C₃-C₁₀)cycloalkyl;
   3-10 membered heterocycloalkyl;
   (C₆-C₁₀)aryl; and
   5-10 membered heteroaryl;
   or R₂ and R₄ together with the carbon atoms carrying them form a (C₃-C₁₀)cycloalkyl group;
wherein said alkyl, cycloalkyl, heterocycloalkyl, aryl and heteroaryl groups are optionally substituted;
R₃ is selected from the group consisting of:
   H;
   (C₃-C₁₀)cycloalkyl;
   3-10 membered heterocycloalkyl;
   (C₆-C₁₀)aryl; and
   5-10 membered heteroaryl;
wherein said cycloalkyl, heterocycloalkyl, aryl and heteroaryl groups are optionally substituted; and
R₄ is selected from the group consisting of: H, —OH, —NRxRy and—C(O)ORz,
Rx, Ry and Rz being independently of each other H or a (C1-C10)alkyl;
or R₂ and R₄ together with the carbon atoms carrying them form a (C₃-C₁₀)cycloalkyl group;
or its pharmaceutically acceptable salt, hydrate or hydrated salt or its polymorphic crystalline structure, racemate, diastereomer or enantiomer.

As used herein, the term “(C₁₋C₂₀)alkyl” or “(C₂-C₂₀)alkyl” means a saturated or unsaturated aliphatic hydrocarbon group which may be straight or branched, having 1 to 20 carbon atoms or 2 to 20 carbon atoms respectively in the chain. Preferred alkyl groups have 1 to 5 carbon atoms in the chain, preferred alkyl groups are in particular methyl or ethyl groups. “Branched” means that one or lower alkyl groups such as methyl, ethyl or propyl are attached to a linear alkyl chain. Alkyl group may be substituted.

As used herein, the term “(C₃-C₁₀)cycloalkyl” means a cyclic, saturated hydrocarbon group having 3 to 10 carbon atoms, wherein any carbon atom capable of substitution may be substituted by a substituent. In particular, cycloalkyl groups are cyclopropyl or cyclohexyl groups.

As used herein, the term “3-10 membered heterocycloalkyl” means a cyclic, saturated hydrocarbon group having 3 to 10 carbon atoms and wherein one or more carbon atom(s) are replaced by one or more heteroatom(s) such as nitrogen atom(s), oxygen atom(s) and sulfur atom(s); for example 1 or 2 nitrogen atom(s), 1 or 2 oxygen atom(s), 1 or 2 sulfur atom(s) or a combination of different heteroatoms such as 1 nitrogen atom and 1 oxygen atom. Any ring atom capable of substitution may be substituted by a substituent. Preferred 3-10 membered heterocycloalkyl are furan, thiophene, nitrogen rings such as pyrrole or pyrazole or fluorophenyl rings.

As used herein, the term “(C₆-C₁₀)aryl” refers to an aromatic monocyclic, bicyclic, or tricyclic hydrocarbon ring system wherein any ring atom capable of substitution may be substituted by a substituent. Examples of aryl moieties include, but are not limited to, phenyl.

As used herein, the term “5-10 membered heteroaryl” refers to an aromatic monocyclic, bicyclic, or tricyclic hydrocarbon ring system, wherein any ring atom capable of substitution may be substituted by a substituent and wherein one or more carbon atom(s) are replaced by one or more heteroatom(s) such as nitrogen atom(s), oxygen atom(s) and sulfur atom(s); for example 1 or 2 nitrogen atom(s), 1 or 2 oxygen atom(s), 1 or 2 sulfur atom(s) or a combination of different heteroatoms such as 1 nitrogen atom and 1 oxygen atom. Preferred heteroaryl groups are thienyl, pyridyl, pyrimydyl and oxazyl groups, more preferably thienyl group.

As used herein, the term “halogen” refers to the atoms of the group 17 of the periodic table and includes in particular fluorine, chlorine, bromine, and iodine atoms, more preferably fluorine, chlorine and bromine atoms, for example fluorine.

As used herein, the term “optionally substituted”, it may be meant that the alkyl, cycloalkyl, heterocycloalkyl, aryl and heteroaryl groups of the compounds of the invention are optionally substituted by one or more substituent(s) selected from the group consisting of: —OH, halogen atom, —C(O)OH, —(C₁-C₁₀)alkyl, —(C₁-C₁₀)alkoxy, and —NR₇R₈ group, wherein R₇ and R₈ are independently of each other selected from (C₁-C₁₀)alkyl or H. Preferably, said alkyl, cycloalkyl, heterocycloalkyl, aryl and heteroaryl groups are optionally substituted by one or more halogen atom(s), more preferably by a fluorine atom.

As used herein, the term “thieno[2,3-b]pyridine derivatives” refer to compounds derived from the following chemical structure:

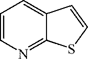

The compounds herein described may have asymmetric centers. Compounds of the present invention containing an asymmetrically substituted atom may be isolated in optically active or racemic forms. It is well-known in the art how to prepare optically active forms, such as by resolution of racemic forms or by synthesis from optically active starting materials. All chiral, diastereomeric, racemic forms and all geometric isomeric forms of a compound are intended, unless the stereochemistry or the isomeric form is specifically indicated.

As used herein, the term “pharmaceutically acceptable salt” refers to salts which retain the biological effectiveness and properties of the compounds of the invention and which are not biologically or otherwise undesirable. Pharmaceutically acceptable acid addition salts may be prepared from inorganic and organic acids, while pharmaceutically acceptable base addition salts can be prepared from inorganic and organic bases. For a review of pharmaceutically acceptable salts see Berge, et al. ((1977) J. Pharm. Sd, vol. 66, 1). For example, the salts include those derived from inorganic acids such as hydrochloric, hydrobromic, sulfuric, sulfamic, phosphoric, nitric, and the like, as well as salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicyclic, sulfanilic, fumaric, methanesulfonic, and toluenesulfonic acid and the like.

A preferred compound of the present invention consists of a compound having the above formula (I), wherein:
R₁ is selected from the group consisting of:
   (C₂-C₂₀)alkyl;
   (C₃-C₁₀)cycloalkyl;
   3-10 membered heterocycloalkyl;
   (C₆-C₁₀)aryl; and
   5-10 membered heteroaryl;
wherein said alkyl, cycloalkyl, heterocycloalkyl, aryl and heteroaryl groups are optionally substituted;
R₂ is selected from the group consisting of:
   H;
   (C₁-C₂₀)alkyl;
   (C₃-C₁₀)cycloalkyl;
   (C₆-C₁₀)aryl; and
   5-10 membered heteroaryl;
   or R₂ and R₄ together with the carbon atoms carrying them form a (C₃-C₁₀)cycloalkyl group;
wherein said alkyl, cycloalkyl, aryl and heteroaryl groups are optionally substituted;
R₃ is selected from the group consisting of:
   H;
   (C₃-C₁₀)cycloalkyl;
   3-10 membered heterocycloalkyl;
   (C₆-C₁₀)aryl; and
   5-10 membered heteroaryl;
wherein said cycloalkyl, heterocycloalkyl, aryl and heteroaryl groups are optionally substituted; and
R₄ is selected from the group consisting of: H, —OH, —NRxRy and —C(O)ORz,
Rx, Ry and Rz being independently of each other H or a (C₁-C₁₀)alkyl;
or R₂ and R₄ together with the carbon atoms carrying them form a (C₃-C₁₀)cycloalkyl group; or its pharmaceutically acceptable salt, hydrate or hydrated salt or its polymorphic crystalline structure, racemate, diastereomer or enantiomer.

In some embodiments, in formula (I), R₃ is selected from the group consisting of:
(C₃-C₁₀)cycloalkyl;
3-10 membered heterocycloalkyl;
(C₆-C₁₀)aryl; and
5-10 membered heteroaryl;
wherein said cycloalkyl, heterocycloalkyl, aryl and heteroaryl groups are optionally substituted.

In some embodiments, in formula (I), R₃ is a (C₆-C₁₀)aryl optionally substituted by one or more substituent(s), preferably selected from the group consisting of: (C₁-C₁₀)alkyl and halogen atom. In some embodiments, R₃ is H or a (C₆-C₁₀)aryl optionally substituted by one or more substituent(s) selected from the group consisting of: (C₁-C₁₀)alkyl and halogen atom. In some embodiments, R₃ is H or a phenyl optionally substituted by one or more halogen atom(s). In some embodiments, R₃ is a phenyl optionally substituted, preferably by one or more halogen atom(s).

In some embodiments, in formula (I), R₁ is selected from the group consisting of:
H;
(C₆-C₁₀)aryl; and
5-10 membered heteroaryl;
wherein said aryl and heteroaryl groups are optionally substituted by one or more substituent(s) selected from the group consisting of —NR₇R₈, (C₁-C₁₀)alkyl and halogen atom; wherein R₇ and R₈ are independently of each other selected from (C₁-C₁₀)alkyl or H.

In some embodiments, in formula (I), R₁ is H or a (C₆-C₁₀)aryl optionally substituted by one or more substituent(s), for example by substituents selected from the group consisting of: (C₁-C₁₀)alkyl, halogen atom and a —NR₇R₈ group; wherein R₇ and R₈ are independently of each other selected from (C₁-C₁₀)alkyl or H. In some embodiments, R₁ is H or a phenyl optionally substituted by one or more halogen atom(s), for example by one fluorine atom, preferably in the para position.

In some embodiments, R₁ is selected from the group consisting of:
(C₆-C₁₀)aryl; and
5-10 membered heteroaryl;
wherein said aryl and heteroaryl groups are optionally substituted by one or more substituent(s) selected from the group consisting of: (C₁-C₁₀)alkyl, halogen atom and a —NR₇R₈ group; wherein R₇ and R₈ are independently of each other selected from (C₁-C₁₀)alkyl or H.

Preferably, in formula (I), R₁ is a (C₆-C₁₀)aryl optionally substituted by one or more substituent(s), for example by substituents selected from the group consisting of: (C₁-C₁₀)alkyl, halogen atom and a —NR₇R₈ group; wherein R₇ and R₈ are independently of each other selected from (C₁-C₁₀)alkyl or H. In some embodiments, R₁ is a phenyl optionally substituted by one or more halogen atom(s), for example by one fluorine atom, preferably in the para position.

In some embodiments, in formula (I), R₂ is selected from the group consisting of:
H;
(C₁-C₂₀)alkyl;
(C₆-C₁₀)aryl; and
5-10 membered heteroaryl;
or R₂ and R₄ together with the carbon atoms carrying them form a (C₃-C₁₀)cycloalkyl group; wherein said alkyl, cycloalkyl, aryl and heteroaryl groups are optionally substituted by one or more substituent(s) selected from the group consisting of: (C₁-C₁₀)alkyl and halogen atom.

In some embodiments, in formula (I), R₂ is selected from the group consisting of: (C₁-C₁₀)alkyl, and 5-6 membered heteroaryl group or R₂ and R₄ together with the carbon atoms carrying them form a (C₃-C₆)cycloalkyl group; wherein said alkyl, cycloalkyl, and heteroaryl groups are optionally substituted, preferably by one or more substituent(s) selected from the group consisting of: (C₁-C₁₀)alkyl and halogen atom.

In some embodiments, in formula (I), R₂ is selected from the group consisting of 5-6 membered heteroaryl groups, said heteroaryl groups being optionally substituted, preferably by one or more substituent(s) selected from the group consisting of: (C₁-C₁₀)alkyl and halogen atom. In some embodiments, R₂ is a thienyl group.

In some embodiments, in formula (I), R₂ is selected from the group consisting of: (C₁-C₁₀)alkyl and a thienyl ring or R₂ and R₄ together with the carbon atoms carrying them form a (C₅-C₆)cycloalkyl group such as a cyclohexyl group.

In some embodiments, in formula (I), R₃ is a (C₆-C₁₀)aryl optionally substituted by one or more substituent(s) preferably selected from the group consisting of: (C₁-C₁₀)alkyl and halogen atom.

In some embodiments, in formula (I), R₄ is selected from the group consisting of: H, —OH, —NH₂ and —C(O)OH or R₂ and R₄ together with the carbon atoms carrying them form a (C₃-C₁₀)cycloalkyl group.

In some embodiments, in formula (I), R₄ is H or R₂ and R₄ together with the carbon atoms carrying them form a (C₅-C₆)cycloalkyl group. Preferably, R₄ is H.

In some embodiments, in formula (I), R₄ is H or R₂ and R₄ together with the carbon atoms carrying them form a (C₅-C₆)cycloalkyl group.

In some embodiments, in formula (I), R₁ is a phenyl group and/or R₂ is a thienyl group, said phenyl and thienyl groups being optionally substituted.

In some embodiments, in formula (I), at least one of R₁ and R₂ is a (C₆-C₁₀)aryl group or a 5-10 membered heteroaryl group.

In some embodiments, the compound of the present invention has the formula (I), wherein:
R₁ is selected from the group consisting of:
   (C₂-C₂₀)alkyl;
   (C₃-C₁₀)cycloalkyl;
   3-10 membered heterocycloalkyl;
   (C₆-C₁₀)aryl; and
   5-10 membered heteroaryl;
wherein said alkyl, cycloalkyl, heterocycloalkyl, aryl and heteroaryl groups are optionally substituted; and
R₂ is selected from the group consisting of:
   H;
   (C₁-C₂₀)alkyl;
   (C₃-C₁₀)cycloalkyl;
   (C₆-C₁₀)aryl; and
   5-10 membered heteroaryl;
or R₂ and R₄ together with the carbon atoms carrying them form a (C₃-C₁₀)cycloalkyl group; wherein said alkyl, cycloalkyl, aryl and heteroaryl groups are optionally substituted.

In some embodiments, the compound of the present invention has the following formula (II):

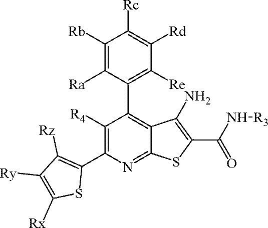

wherein:
Ra, Rb, Rc, Rd, Re, Rx, Ry and Rz are selected among the group consisting of: H, —OH, halogen atom, —C(O)OH, (C₁-C₁₀)alkyl, (C₁-C₁₀)alkoxy, and —NR₅R₆,
wherein R₅ and R₆ are independently of each other selected from (C₁-C₁₀)alkyl or H;
R₄ is selected from the group consisting of H, —OH, —NH₂ and —C(O)OH; and
R₃ is as defined in formula (II) above.

In some embodiments, in formula (II), Ra, Rb, Rc, Rd, Re, Rx, Ry and Rz are selected among H, halogen atom or (C₁-C₁₀)alkyl.

In some embodiments, in formula (II), Rx, Ry and Rz are H and/or Ra, Rb, Rd and Re are H.

Preferably, in formula (II), Rc is H or an halogen atom, for example a fluorine atom.

In some embodiments, the compound of the present invention has one of the following formulae:

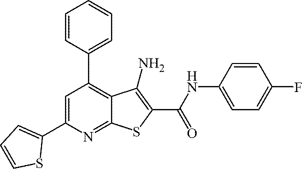 [SMILES: Nc1c(C(=O)Nc2ccc(F)cc2)sc2nc(-c3cccs3)cc(-c3ccccc3)c12]

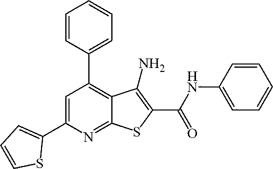 [SMILES: Nc1c(C(=O)Nc2ccccc2)sc2nc(-c3cccs3)cc(-c3ccccc3)c12]

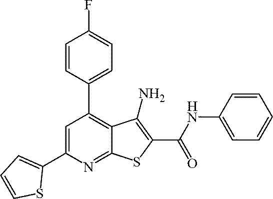 [SMILES: Nc1c(C(=O)Nc2ccccc2)sc2nc(-c3cccs3)cc(-c3ccc(F)cc3)c12]

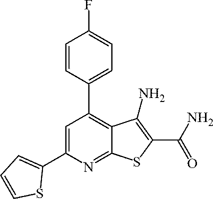 [SMILES: NC(=O)c1sc2nc(-c3cccs3)cc(-c3ccc(F)cc3)c2c1N]

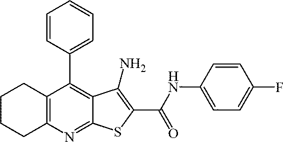 [SMILES: Nc1c(C(=O)Nc2ccc(F)cc2)sc2nc3c(c(-c4ccccc4)c12)CCCC3]

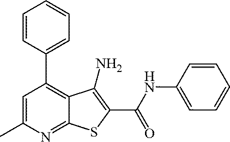 [SMILES: Cc1cc(-c2ccccc2)c2c(N)c(C(=O)Nc3ccccc3)sc2n1]

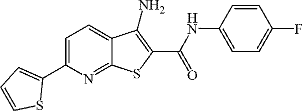 [SMILES: Nc1c(C(=O)Nc2ccc(F)cc2)sc2nc(-c3cccs3)ccc12]

In some embodiments, the compound of the present invention has the following formula:

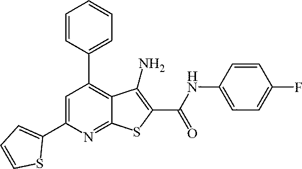 [SMILES: Nc1c(C(=O)Nc2ccc(F)cc2)sc2nc(-c3cccs3)cc(-c3ccccc3)c12]

As used herein, the term “coronavirus” has its general meaning in the art and refers to any member of members of the Coronaviridae family. Coronavirus is a virus whose genome is plus-stranded RNA of about 27 kb to about 33 kb in length depending on the particular virus. The virion RNA has a cap at the 5′ end and a poly A tail at the 3′ end. The length of the RNA makes coronaviruses the largest of the RNA virus genomes. In particular, coronavirus RNAs encode: (1) an RNA-dependent RNA polymerase; (2) N-protein; (3) three envelope glycoproteins; plus (4) three non-structural proteins. These coronaviruses infect a variety of mammals and birds. In humans, they cause respiratory infections (common), enteric infections (mostly in infants >12 mo.), and possibly neurological syndromes. Coronaviruses are transmitted by aerosols of respiratory secretions. Coronaviruses are exemplified by, but not limited to, human enteric coV (ATCC accession # VR-1475), human coV 229E (ATCC accession # VR-740), human coV OC43 (ATCC accession # VR-920), Middle East respiratory syndrome-related coronavirus (MERS-Cov) and SARS-coronavirus (Center for Disease Control), in particular SARS-Cov1 and SARS-Cov2.

According to the present invention, the compound of the present invention is particularly suitable for inhibiting the replication of the coronavirus as demonstrated in EXAMPLE.

In particular, the compound of the present invention is suitable for the treatment of Severe Acute Respiratory Syndrome (SARS). More particularly, the compound of the present invention is suitable for the treatment of COVID-19.

In some embodiments, the subject can be human or any other animal (e.g., birds and mammals) susceptible to coronavirus infection (e.g. domestic animals such as cats and dogs; livestock and farm animals such as horses, cows, pigs, chickens, etc.). Typically said subject is a mammal including a non-primate (e.g., a camel, donkey, zebra, cow, pig, horse, goat, sheep, cat, dog, rat, and mouse) and a primate (e.g., a monkey, chimpanzee, and a human). In some embodiments, the subject is a non-human animal. In some embodiments, the subject is a farm animal or pet. In some embodiments, the subject is a human. In some embodiments, the subject is a human infant. In some embodiments, the subject is a human child. In some embodiments, the subject is a human adult. In some embodiments, the subject is an elderly human. In some embodiments, the subject is a premature human infant.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular interval, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

In some embodiment, the compound of the present invention is administered to the subject in combination with at least one other therapeutic agent, preferably in combination with at least one other antiviral agent, more preferably in combination with at least one other antiviral agent selected from the group consisting of remdesivir, lopinavir, ritonavir, hydroxycholoroquine, and chloroquine.

As used herein, the term “therapeutically effective amount” of the compound of the present invention is meant a sufficient amount of the compound to treat a coronavirus infection at a reasonable benefit/risk ratio applicable to any medical treatment. It will be understood, however, that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; activity of the specific compound employed; the specific composition employed, the age, body weight, general health, sex and diet of the patient; the time of administration, route of administration, and rate of excretion of the specific compound employed; the duration of the treatment; drugs used in combination with the specific agonist employed; and like factors well known in the medical arts. For example, it is well known within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the patient to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

Typically, the compound of the present invention may be combined with pharmaceutically acceptable excipients, and optionally sustained-release matrices, such as biodegradable polymers, to form therapeutic compositions.

As used herein, the term “pharmaceutically” or “pharmaceutically acceptable” refers to molecular entities and compositions that do not produce an adverse, allergic or other untoward reaction when administered to a mammal, especially a human, as appropriate.

A pharmaceutically acceptable carrier or excipient refers to a non-toxic solid, semi-solid or liquid filler, diluent, encapsulating material or formulation auxiliary of any type. In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Galenic adaptations may be done for specific delivery in the small intestine or colon. Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol ; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising the compound of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The compound of the invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifusoluble agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject. The compound of the invention may be formulated within a therapeutic mixture to comprise about 0.0001 to 1.0 milligrams, or about 0.001 to 0.1 milligrams, or about 0.1 to 1.0 or even about 10 milligrams per dose or so. Multiple doses can also be administered. In addition to compound formulated for parenteral administration, such as intravenous or intramuscular injection, other pharmaceutically acceptable forms include, e.g. tablets or other solids for oral administration; liposomal formulations ; time release capsules ; and any other form currently used.

The invention will be further illustrated by the following figures and examples. However, these examples and figures should not be interpreted in any way as limiting the scope of the present invention.

### FIGURES

FIG. 1. Inhibition of SARS-CoV-2 replication in VeroE6 treated with AM-001. VeroE6 cells pretreated for one hour with the indicated concentrations of AM-001 were infected at a multiplicity of infection of 0.001. After one hour of virus inoculation, the cell culture medium was removed and replaced with cell culture medium containing AM-001 at the indicated concentrations. 24 hours post-infection, viral RNA was extracted from the supernatants of infected cells and subjected to RT-qPCR using primers and probe specific for the E gene. The dotted line indicated 90% viral inhibition.

FIG. 2. Inhibition of SARS-CoV-2 replication in human lung epithelial Calu-3 cells pretreated with AM-001. Calu-3 cells pretreated for two hours with the indicated concentrations of AM-001 were infected at a multiplicity of infection of 0.001. After one hour of virus inoculation, the cell culture medium was removed and replaced with cell culture medium containing AM-001 at the indicated concentrations or Remdesivir at 6 µM. (A) 24 hours post-infection, viral RNA was extracted from the supernatants of infected cells and subjected to RT-qPCR using primers and probe specific for the E gene. (B) 24 hours post-infection, infectious particles in the supernatant were titrated using the TCID₅₀ method.

FIG. 3. Analysis of cellular viability in human lung epithelial Calu-3 cells treated with AM-001. Calu-3 cells treated for 24 hours with the indicated concentrations of AM-001. Cellular viability was measured with the Cell Proliferation Kit I (MTT) (Roche Applied Science, Indianapolis, IN) according to the manufacturer’s protocol.

FIG. 4 Evaluation of the antiviral properties of AM-001 against SARS-CoV-2 replication in human lung epithelial Calu-3 cells upon pre-treatment, treatment or post-treatment. Calu-3 cells were treated and infected as described in Material and Methods. 24 hours post-infection, infectious particles in the supernatant were titrated using the TCID₅₀ method.

### EXAMPLE 1

### Material & Methods

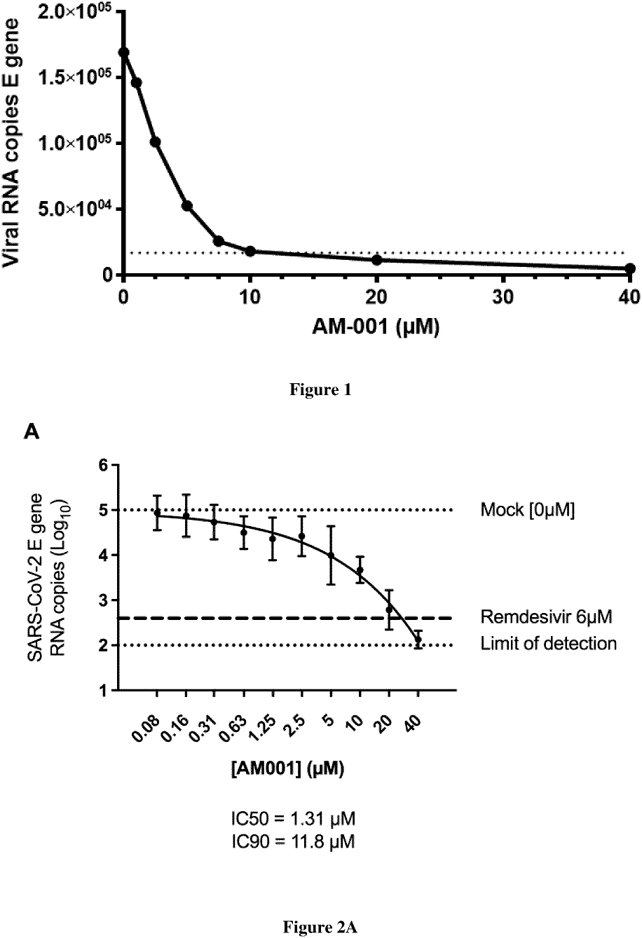

VeroE6 cells (ATCC CRL-1586) were cultured in Dulbecco modified Eagle’s minimal essential medium (DMEM) containing 10% fecal calf serum (FCS). The SARS-CoV-2 virus used in these studies was isolated from a nasal swab kindly provided by the Toulouse hospital (CHU Toulouse Purpan, France) following two passages in VeroE6 cells. For the SARS-CoV-2 infection, the cells were plated in 6-well plates at a density of 2.10⁵ cells per well. The next day, the cells were pretreated for one hour with AM-001 at different concentrations. Following one hour pretreatment, the medium was removed and the cells were infected at a multiplicity of infection of 0.001 with SARS-CoV-2 in DMEM containing 2% FCS (infection medium). After one hour of virus inoculation, the cell culture medium was removed and replaced with infection medium containing AM-001 at the different concentrations. 24 hours post-infection, viral RNA was extracted from the supernatants of infected cells and subjected to RT-qPCR using primers and probe specific for the E gene. The result is depicted in FIG. 1.

### EXAMPLE 2

### Material & Methods

### Cell Culture

Human lung epithelial cells Calu-3 (ATCC HTB-55) were cultured in Dulbecco’s Modified Eagle Medium: Nutrient Mixture F-12 (DMEM:F12) containing 20% fecal calf serum (FCS) and GlutaMAX™ Supplement 1X (Gibco).Vero E6 cells (ATCC CRL-1586) were cultured in Dulbecco modified Eagle’s minimal essential medium (DMEM) containing 10% fecal calf serum (FCS). The SARS-CoV-2 virus used in these studies was isolated from a nasal swab kindly provided by the Toulouse hospital (CHU Toulouse Purpan, France) following two passages in Vero E6 cells. For the SARS-CoV-2 infection and evaluation of the antiviral properties of AM-001, Calu-3 cells were plated 48 hours before treatment and infection in 12-wells plates at a density of 1.10⁶ cells per well.

### Pretreatment

Cells were pretreated for two hours with AM-001 at different concentrations or with Remdesivir at 6 µM. Following pretreatment, the medium was removed and the cells were infected at a multiplicity of infection of 0.001 with SARS-CoV-2 in medium containing 2% FCS (infection medium) and AM-001 at the different concentrations. After one hour of virus inoculation, cells were washed twice with PBS and the cell culture medium was replaced with infection medium containing AM-001 at the different concentrations or with Remdesivir at 6 µM. 24 hours post-infection, viral RNA was extracted from the supernatants of infected cells and subjected to RT-qPCR using primers and probe specific for the E gene to quantify viral RNA in the supernatants. 24 hours post-infection, infectious virus was also titrated from the supernatants of infected cells by the tissue culture infectious dose 50% (TCID₅₀) method on Vero E6 cells.

### Treatment

Cells were infected at a multiplicity of infection of 0.001 with SARS-CoV-2 in medium containing 2% FCS (infection medium) and AM-001 at the different concentrations or with Remdesivir at 6 µM. After one hour of virus inoculation, cells were washed twice with PBS and the cell culture medium was replaced with infection medium containing AM-001 at the different concentrations. 24 hours post-infection, viral RNA was extracted from the supernatants of infected cells and subjected to RT-qPCR using primers and probe specific for the E gene to quantify viral RNA in the supernatants. 24 hours post-infection, infectious virus was also titrated from the supernatants of infected cells by the tissue culture infectious dose 50% (TCID₅₀) method on VeroE6 cells.

### Post-Treatment

Cells were infected at a multiplicity of infection of 0.001 with SARS-CoV-2 in medium containing 2% FCS (infection medium). After one hour of virus inoculation, cells were washed twice with PBS and the cell culture medium was replaced by fresh infection medium. Four hours post-infection, the cell culture medium was removed and replaced by infection medium containing AM-001 at the different concentrations or with Remdesivir at 6 µM. 24 hours post-infection, viral RNA was extracted from the supernatants of infected cells and subjected to RT-qPCR using primers and probe specific for the E gene to quantify viral RNA in the supernatants. 24 hours post-infection, infectious virus was also titrated from the supernatants of infected cells by the tissue culture infectious dose 50% (TCID₅₀) method on VeroE6 cells.

### Cellular Viability Assay

Calu-3 cells were plated 48 hours before treatment in 96-wells plates at a density of 1.10⁵ cells per well. After 24 hours of treatment with the different concentrations of AM-001, cells viability was measured using the Cell Proliferation Kit I (MTT) (Roche Applied Science, Indianapolis, IN) according to the manufacturer’s protocol.

### Results

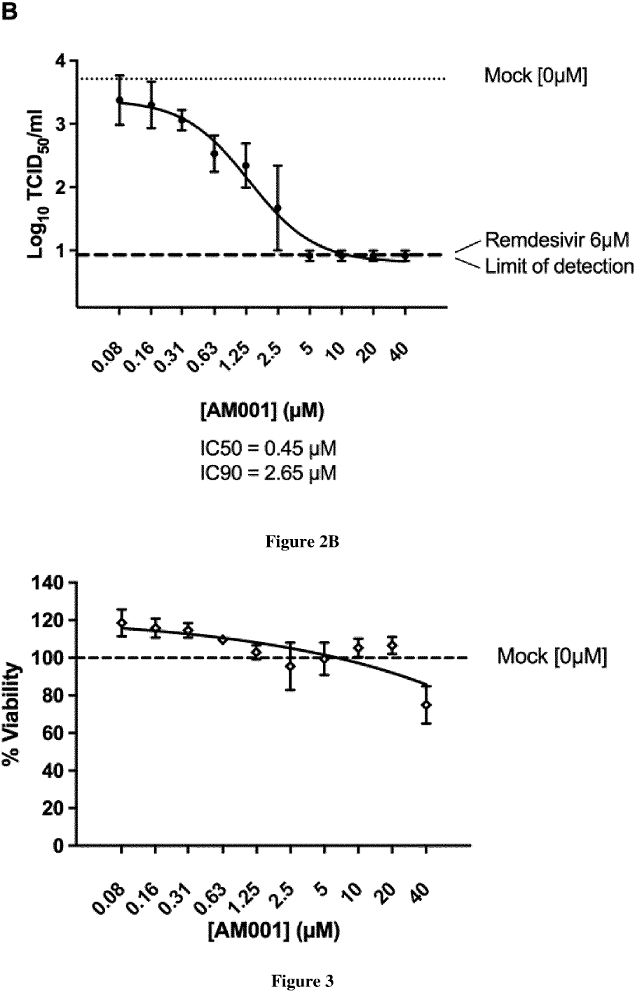

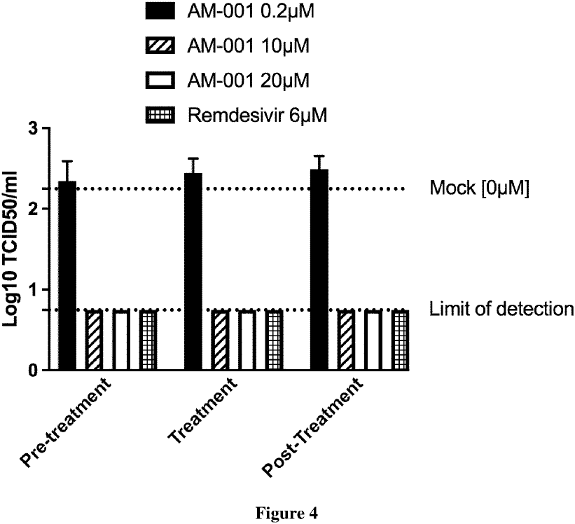

AM-001 pre-treatment inhibit SARS CoV-2 replication (FIGS. 2A-2B). AM-001 has no detected cellular toxicity (FIG. 3). When used at concentration ≥ 10 µM, AM-001 totally blocks infectious virus production even when given 4 h post-infection in human lung epithelial Calu3 cells (FIG. 4). The reported antiviral potency of AM-001 is similar to the anti-viral molecule Remdesivir (FIGS. 2A-2B and 4).

### REFERENCES

Throughout this application, various references describe the state of the art to which this invention pertains. The disclosures of these references are hereby incorporated by reference into the present disclosure.

1. A method of treating a coronavirus infection in a subject in need thereof comprising administrating to the subject a therapeutically effective amount of a compound having the following formula (I) 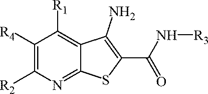
wherein:
R₁ is selected from the group consisting of:
H;
(C₂-C₂₀)alkyl;
(C₃-C₁₀)cycloalkyl;
3-10 membered heterocycloalkyl;
(C₆-C₁₀)aryl; and
5-10 membered heteroaryl;
wherein said alkyl, cycloalkyl, heterocycloalkyl, aryl and heteroaryl groups are optionally substituted;
R₂ is selected from the group consisting of:
H;
(C₁-C₂₀)alkyl;
(C₃-C₁₀)cycloalkyl;
3-10 membered heterocycloalkyl;
(C₆-C₁₀)aryl; and
5-10 membered heteroaryl;
or R₂ and R₄ together with the carbon atoms carrying them form a (C₃-C₁₀)cycloalkyl group;
wherein said (C₂-C₂₀) alkyl and said (C₁-C₂₀₎ alkyl, said (C₃-C₁₀) cycloalkyl, said 3-10 membered heterocycloalkyl, said (C₆-C₁₀) aryl and said 5-10 membered heteroaryl groups are optionally substituted;
R3 is selected from the group consisting of:
H;
(C₃-C₁₀)cycloalkyl;
3-10 membered heterocycloalkyl;
(C₆-C₁₀)aryl; and
5-10 membered heteroaryl;
wherein said (C₃-C₁₀) cycloalkyl, said 3-10 membered heterocycloalkyl, said (C₆-C₁₀) aryl and said 5-10 membered heteroaryl groups are optionally substituted; and
R₄ is selected from the group consisting of: H, —OH, —NRxRy and —C(O)ORz,
Rx, Ry and Rz being independently of each other H or a (C₁-C₁₀)alkyl;
or R₂ and R₄ together with the carbon atoms carrying them form a (C₃-C₁₀)cycloalkyl group;
or its pharmaceutically acceptable salt, hydrate or hydrated salt or its polymorphic crystalline structure, racemate, diastereomer or enantiomer.

2. The method of claim 1 wherein in formula (I), R₃ is selected from the group consisting of:
(C₃-C₁₀)cycloalkyl;
3-10 membered heterocycloalkyl;
(C₆-C₁₀)aryl; and
5-10 membered heteroaryl;
and wherein said (C₃-C₁₀) cycloalkyl, said 3-10 membered heterocycloalkyl, said (C₆-C₁₀₎ aryl and said 5-10 membered heteroaryl groups are optionally substituted.

3. The method of claim 1, wherein, in formula (I), R₁ is selected from the group consisting of:
H;
(C₆-C₁₀)aryl; and
5-10 membered heteroaryl;
wherein said (C₆-C₁₀) aryl and said 5-10 membered heteroaryl groups are optionally substituted by one or more substituent(s) selected from the group consisting of —NR7R8, (C₁-C₁₀)alkyl and halogen atom; wherein R7 and R8 are independently of each other selected from (C₁-C₁₀)alkyl and H.

4. The method of claim 1, wherein, in formula (I), R₂ is selected from the group consisting of:
H;
(C₁-C₂₀)alkyl;
(C₆-C₁₀)aryl; and
5-10 membered heteroaryl;
or R₂ and R₄ together with the carbon atoms carrying them form a (C₃-C₁₀)cycloalkyl group;
wherein said (C₁-C₂₀₎ alkyl, said (C₃-C₁₀) cycloalkyl, said (C₆-C₁₀) aryl and said 5-10 membered heteroaryl groups are optionally substituted by one or more substituent(s) selected from the group consisting of: (C₁-C₁₀)alkyl and halogen atom.

5. The method of claim 1 wherein in formula (I), R₃ is a (C₆-C₁₀)aryl optionally substituted by one or more substituent(s).

6. The method of claim 1 wherein, in formula (I), R₄ is H or R₂ and R₄ together with the carbon atoms carrying them form a (C₅-C₆)cycloalkyl group.

7. The method of claim 1 wherein, in formula (I), R₁ is a phenyl group and/or R₂ is a thienyl group, said phenyl and thienyl groups being optionally substituted.

8. The method of claim 1 wherein, in formula (I):
R₁ is selected from the group consisting of:
(C₂-C₂₀)alkyl;
(C₃-C₁₀)cycloalkyl;
3-10 membered heterocycloalkyl;
(C₆-C₁₀)aryl; and
5-10 membered heteroaryl;
wherein said (C₂-C₂₀) alkyl, said (C₃-C₁₀) cycloalkyl, said 3-10 membered heterocycloalkyl, said (C₆-C₁₀) aryl and said 5-10 membered heteroaryl groups are optionally substituted; and
R2 is selected from the group consisting of:
H;
(C₁-C₂₀)alkyl;
(C₃-C₁₀)cycloalkyl;
(C₆-C₁₀)aryl; and
5-10 membered heteroaryl;
or R₂ and R₄ together with the carbon atoms carrying them form a (C₃-C₁₀)cycloalkyl group;
wherein said (C₁-C₂₀) alkyl, said (C₃-C₁₀) cycloalkyl, said (C₆-C₁₀) aryl and said 5-10 membered heteroaryl groups are optionally substituted.

9. The method of claim 1 wherein the compound is characterized by the following formula (II): 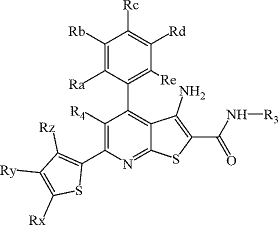
wherein Ra, Rb, Rc, Rd, Re, Rx, Ry and Rz are selected among the group consisting of: H, —OH, halogen atom, —C(O)OH, (C₁-C₁₀)alkyl, (C₁-C₁₀)alkoxy, and NR5R₆,
wherein R₅ and R₆ are independently of each other selected from (C₁-C₁₀)alkyl or H;
R₄ is selected from the group consisting of H, —OH, —NH2 and —C(O)OH; and
R₃ is selected from the group consisting of:
H;
(C₃-C₁₀)cycloalkyl;
3-10 membered heterocycloalkyl;
(C₆-C₁₀)aryl; and
5-10 membered heteroaryl;
wherein said (C₃-C₁₀) cycloalkyl, said 3-10 membered heterocycloalkyl, said (C₆-C₁₀) aryl and said 3-10 membered heteroaryl groups are optionally substituted.

10. The method of claim 1 wherein the compound has one of the following formulae: 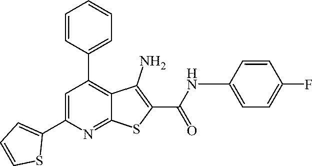 [SMILES: Nc1c(C(=O)Nc2ccc(F)cc2)sc2nc(-c3cccs3)cc(-c3ccccc3)c12] 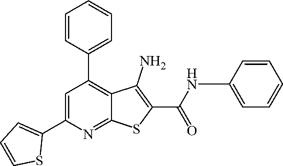 [SMILES: Nc1c(C(=O)Nc2ccccc2)sc2nc(-c3cccs3)cc(-c3ccccc3)c12] 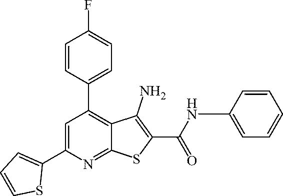 [SMILES: Nc1c(C(=O)Nc2ccccc2)sc2nc(-c3cccs3)cc(-c3ccc(F)cc3)c12] 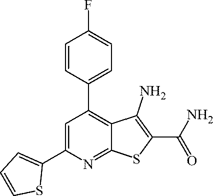 [SMILES: NC(=O)c1sc2nc(-c3cccs3)cc(-c3ccc(F)cc3)c2c1N] 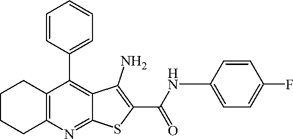 [SMILES: Nc1c(C(=O)Nc2ccc(F)cc2)sc2nc3c(c(-c4ccccc4)c12)CCCC3] 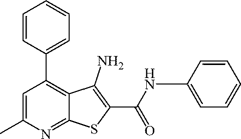 [SMILES: Cc1cc(-c2ccccc2)c2c(N)c(C(=O)Nc3ccccc3)sc2n1] 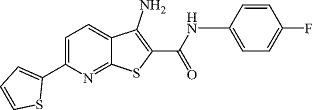 [SMILES: Nc1c(C(=O)Nc2ccc(F)cc2)sc2nc(-c3cccs3)ccc12]
.

11. The method of claim 1 wherein the compound has the following formula: 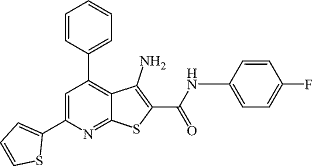 [SMILES: Nc1c(C(=O)Nc2ccc(F)cc2)sc2nc(-c3cccs3)cc(-c3ccccc3)c12]
.

12. The method of claim 1 wherein the coronavirus is the SARS-Cov-2.

13. The method of claim 5 wherein the one or more substituent(s) are (C₁-C₁₀)alkyl or halogen atom.
